(19) United States
(12) Patent Application Publication
(10) Pub. No.: US 2025/0031690 A1
(43) Pub. Date: Jan. 30, 2025

(54) Medium for the in vitro transportation and storage of cells

(71) Applicant: LUXEMBOURG INSTITUTE OF SCIENCE AND TECHNOLOGY, ESCH-SUR-ALZETTE (LU)

(72) Inventors: Elisa MOSCHINI, LUXEMBOURG (FR); Christos SOUKOULIS, LUXEMBOURG (FR); Tommaso SERCHI, KIRSCH-LES-SIERCK (FR)

(21) Appl. No.: 18/268,597

(22) PCT Filed: Dec. 21, 2021
(86) PCT No.: PCT/EP2021/087010

§ 371 Date: Jun. 20, 2023

(30) Foreign Application Priority Data
Dec. 21, 2020 (LU) 102338

(51) Int. Cl. A01N 1/02; C08J 3/075; C12N 5/00

(52) U.S. Cl. CPC A01N 1/0231; C08J 3/075; C12N 5/0068; C12N 2533/78

(57) ABSTRACT

The invention relates to an in vitro storing and/or transporting of cells, comprising: —contacting the cells with an aqueous thermo-reversible gelling composition, in a liquid form and comprising: (A) a thermo-reversible gelling polysaccharide selected from carrageenan, gellan gum and konjac gum; and (B) carboxymethyl cellulose as a thickening agent-thermo-gelling the composition by decreasing the temperature; —storing and/or transporting the in vitro cultivated cells embedded in the obtained hydrogel as obtained The invention also relates to the specific thermo-reversible gelling composition used in the process:

### FIELD OF THE INVENTION

The instant invention relates to the in vitro transportation and storage of cells, and more specifically to gelling compositions useful as a suitable medium for cultivated and/or isolated cells during transportation or storage operations.

### BACKGROUND OF THE INVENTION

When transporting in vitro cultivated or isolated cells, the cells have to be preserved in a proper medium, allowing their integrity and viability during operations such as long-distance shipping and/or intermediate storing.

To this end, methods of transport in cryovials on dry ice or in liquid nitrogen have been described and commonly used, but this cryopreserved transport induces high costs, in addition to issues due to the hazardous nature of dry ice and liquid nitrogen. Besides, these methods need long periods of adaptation of the cells after their transport (generally a few days or weeks) before they can be used.

Alternatively, a transportation of the cells in a cultured state is possible, typically with adherent cells or free-floating cells surrounded by a liquid culture medium, but, it is then necessary to be extremely careful with the transport conditions that may dramatically affect the integrity of the cells and/or their functional capacities, such as their adhesion abilities.

Maintaining the integrity and the functional capacities of the cells is especially important for in vitro cultured cells used as models (for example for modelling genetic, biochemical, metabolic or physiological processes) and more generally for cells cultured on specific supports, and for co-cultured cells especially when placed on asymmetrical supports such as Transwell-type supports. In that case not only the integrity of each cell has to be preserved, but also the whole structure of the culture, in particular when it is intended to act as a model.

Methods have been developed that avoid the aforementioned problems, wherein the cultured cells are transported in a semi-solid medium, typically a gel.

In this connection, reference may especially be made to the method described in the patent U.S. Pat. No. 8,900,842, that makes use of a gelling composition based on gelatin for storing and/or transporting in vitro organized cell cultures on an asymmetric support. In U.S. Pat. No. 8,900,842 the organized cell cultures coated with a culture medium including the gelatin and the gelatin are then gelled at a temperature of between 15 and 25° C., which allows the transport/storage in the same range of temperature. This method is especially interesting since the obtained gelification (gelling) is thermo-reversible. Namely, after the transportation/storage a liquefaction may be easily obtained, simply by incubating the solidified composition including the cells at 37° C. which induces a liquefaction of the gelatin. The liquefaction allows the washing of the composition used for the transportation/storage and its replacement by a culture medium.

Other methods making use of gels for transporting/storing cells have been described, but they are generally of less interest, since the gels are not directly reversible for recovering the cells.

As an example, the method of patent U.S. Pat. No. 10,655,120 makes use of a hydrogel, typically an irreversible hydrogel based on alginate, that needs to be chemically disintegrated or dissolved, which may be harmful to the cells and to the cell culture architecture in case of complex cultures such as models or co-cultured cells.

Methods using gels based on agarose have also been considered. These gels are thermo-reversible, but the liquefaction (gel-sol process) implies high temperatures not compatible with the preservation of the cells (typically of at least 50° C.). A specific method is described in U.S. Pat. No. 8,709,803 wherein the agarose is used in admixture of an agarase which is deactivated at low temperature (25° C. or less where the mixture of agarose and agarase, once solidified allows cell transport) and reactivated by increasing the temperature by incubation at 37° C., which leads to an enzymatic digestion of the agarose of the gel and induces its liquefaction. In this method, again, an additional compound is needed for recovering the cells.

An issue with the existing methods using a thermo-reversible gel, especially with the method using gelatin disclosed in U.S. Pat. No. 8,900,842, or the use of agarose-agarase mixtures of U.S. Pat. No. 8,709,803, is that they need time, both (i) before the transportation/storage for the formation of the gel (“sol-gel time”); and (ii) after the transportation/storage for the liquefaction of the gel (“gel-sol time”). Each of the sol-gel step and gel-sol step duration is typically of several hours.

### AIM OF THE INVENTION

One aim of the instant invention is to provide a method for the in vitro transport and/or storage of cells, having the benefits of the existing methods making use of thermo-reversible gels, but which necessitate a low working time for forming the gel and then for recovering the cells, especially a lower working time than the total duration of the sol-gel step and gel-sol steps of U.S. Pat. Nos. 8,900,842 and 8,709,803.

In this connection, the invention especially aims at providing a method suitable for the transport or storage of cells cultured or co-cultured on specific, e.g. asymmetrical, supports such as transwell-type supports for example, and more generally for cell cultures having a specific architecture, for example intended to be used as models.

### SUMMARY OF THE INVENTION

The present invention makes use of a specific thermo-reversible gelling composition, herein-referred as the “composition C”. The inventors have now found that, among other advantages which are described hereinafter, this composition C notably allows a suitable in vitro transport and/or storage of cells, in the same way as with the composition based on gelatin which is e.g. disclosed in U.S. Pat. No. 8,900,842, but with a lower total duration of the sol/gel and gel/sol steps.

More precisely, one subject-matter of the invention is a process for the in vitro storing and/or transporting of cells (typically cultivated or isolated and preferably cultivated cells), comprising the following successive steps:
STEP 1: contacting the cells with an aqueous thermo-reversible gelling composition C, said composition C being in a liquid form and comprising, in an aqueous medium:
   (A) a thermo-reversible gelling polysaccharide selected from carrageenan, gellan gum and konjac gum; and
   (B) carboxymethyl cellulose as a thickening agent
STEP 2: thermo-gelling the composition C in contact with the cells as obtained in step 1, by decreasing the temperature, whereby a hydrogel is obtained, embedding the cells;
STEP 3: storing and/or transporting the cells embedded in the hydrogel as obtained in step 2.

In most cases, the process of the invention further comprises the following additional step 4, after step 3:
STEP 4: liquefying the hydrogel by increasing the temperature, whereby the cells are released from the hydrogel

According to another aspect, one specific subject-matter of the invention is the composition C, useful for the aforementioned process, comprising, in an aqueous medium:
(A) a thermo-reversible gelling polysaccharide selected from carrageenan, gellan gum and konjac gum; and
(B) carboxymethyl cellulose as a thickening agent

### DETAILED DESCRIPTION OF THE INVENTION

The composition C according to the invention is specifically based on an aqueous medium, which is generally a homogenous phase based on water which may contain additives such as salts or water soluble solvents. The aqueous medium of composition C typically comprises at least 90% by weight of water based on the total weight of the composition C. Most often, the aqueous medium contains at least 95%, preferably at least 97%, notably at least 98% by weight of water based on the total weight of the composition C.

Typically, the aqueous medium of composition C is a culture medium suitable for the stored and/or transported cells. One advantage of the process of the invention is that virtually any kind of aqueous culture medium may be used as the aqueous medium of composition C. Depending on the nature and concentration of salts present in the medium, the ratio A/B may be adapted and the presence of EDTA should be preferable, but there is no technical limitation as regards the culture medium in composition C.

Typically, the cells to be transported and/or stored according to the invention are embedded in or coated by a culture medium before they are submitted to STEP 1 of the invention, referred herein as an “initial culture medium”. In most cases, even if not strictly needed according to the invention, the process advantageously comprises a medium substitution step wherein all or part of the initial culture medium is replaced by composition C. In that case, STEP 1 may typically be carried out by removing all or part (and typically substantially all) of the initial culture medium and then add a composition C according to the invention. In that case, the aqueous medium of the composition C may typically have the same composition as the initial culture medium, but according to possible variant, the composition may also be different in composition C.

A composition C according to the invention includes, as a main component a thermo-reversible gelling polysaccharide (A), which is selected from carrageenan, gellan gum and konjac gum. This compound (A) especially confers a suitable elasticity to the hydrogel formed in STEP 2 and used in STEP 3.

According to a specific embodiment, the compound (A) present in a composition C according to the invention is a carrageenan. An alternative embodiment makes use of gellan gum, and another specific embodiment uses konjac gum.

According to an especially suitable embodiment, the compound (A) includes—and preferably is—a carrageenan, preferably a kappa-carrageenan, having preferably a viscosity between 5 and 25 mPa·s at 25° C. at 0.3% by weight in water. The solubility of a carrageenan useful according to the invention is typically of about 5 mg/mL in hot water. A suitable kappa-carrageenan according to the invention is for example the kappa carrageenan from red algae available from Sigma Aldrich (corresponding to CAS no. 11114-20-8) or the commercial product Grinsted, CW series, from Dupont.

A second component of the composition C according to the invention is the thickening agent (B), which is a carboxymethyl cellulose. This second compound (B) notably confers a suitable stiffness to the hydrogel formed in STEP 2 and used in STEP 3, which avoids any leakage during the transportation and or storage of the cells. The association of the elasticity to compound (A) and stiffness conferred by component (B) allows the transportation of virtually any kind of cells, even cell cultures with complex architectures for example co-cultured cells on asymmetrical support, with a proper preservation of both the cells and of their properties and also the organization of the cell in the architecture of the culture, which allows long distance shipping.

Preferably, the compound (B) used according to the invention is a carboxymethyl cellulose having a viscosity of between 50 and 200 mPa·s at 25° C. at 4% by weight in water, with typically a solubility in cold water of about 40 mg/mL. Preferably, a carboxymethyl cellulose useful according to the invention has a viscosity lower than 40 mPa·s at 25° C. at 2% by weight in water. Suitable carboxymethyl cellulose includes for example the following commercial products:
carboxymethylcellulose sodium salt (corresponding to CAS no. 9004-32-4) such as the Low Viscosity USP, Spectrum™, from FisherScientific; or the Low Viscosity Carboxymethylcellulose sodium salt from Sigma-Aldrich;
Acqualon CMC 7L2, or Acqualon CMC 7M2F from Dupont.

Notably to obtain suitable elasticity and stiffness, it is generally preferable that, in a composition C according to the invention, the ratio A/B of the total mass of the compound (A) to the total mass of the compound (B) is between 15:85 to 40:60, preferably between 20:80 and 40:60.

Besides, in a composition C as used according to the invention, the total concentration of the compounds (A) and (B), namely the ratio of the sum of the mass of compound (A) plus the mass of compound (B) to the total volume of composition (C) is preferably between 6 to 10 g/L, for example preferably between 8 and 10 g/L.

Typically in a composition C according to the invention, the concentration of compound (A) is from 1.5 to 4 g/L, more preferably from 1.5 to 2.3 g/L. Besides, the concentration of compound (B) is generally from 6 to 8.5 g/L, more preferably from 7.7 to 8.5 g/L.

According to a possible embodiment, a composition (C) according to the invention may further comprise ethylenediaminetetraacetic acid (EDTA) as a chelating agent. This chelating agent may especially be useful for helping the de-gelling process in STEP 4, by chelating the excess of cations optionally derived from the physiological cell metabolism during the transport/storage, that would otherwise lead to aggregation 35 especially when compound (A) is a kappa-carrageenan. When EDTA is used, its content is preferably of less than 1 mM in the composition (C).

One interesting aspect of the invention is that the compounds needed for obtaining the results sought in the scope of the instant invention are not from animal origin. According to an especially preferred embodiment of the invention, the composition C does not contain any substances from animal origin.

This possibility of providing an animal-free gelling composition is another great advantage of the present invention in comparison to the method described in U.S. Pat. No. 8,900,842 that makes use of gelatin like gelatin from porcine skin.

This first constitutes an improvement from the ethical point of view and allows to achieve for example the objectives of the so-called 3Rs principles (Reduction, Refinement, and Replacement of animal use in science). The in vitro transportation and storage method of the invention constitutes in this connection a real improvement and allows to avoid animal sourced products that in vitro systems often still heavily rely on. Especially, the invention allows to avoid the presence of the following animal sourced ingredients (that are preferably not present in the composition used in the composition of the invention): fetal calf serum (FCS), animal-sourced enzymes, collagen, gelatin.

In addition, and independently from the above ethical considerations, the possibility offered by the invention to provide an animal-free technical solution has also more impartial technical advantages. Namely, the absence of animal-sourced compounds may also induce a reduction of the experimental variability that is linked to the use of animals and to the different batches of products, which allows for more controlled products, ensuring best reproducibility and quality of the downstream applications. More important, the absence of animal-sourced compounds reduces the risks of contamination, especially when compared with the use of gelatin.

Since the composition C of the invention allows, if needed, to avoid the uses of any animal-sourced compounds, the composition may advantageously be used within a global process that avoids the use of any animal-sourced compounds. Especially, according to a specific embodiment, the composition C of the invention does not contain and is not used with serum such as fetal calf serum.

In this connection, the culture medium used as the aqueous medium of composition C may be e.g. selected from the following media, that are free from serum:
PneumaCult™-ALI Medium and supplements, from STEMCELL Technologies
PneumaCult™-Ex Medium and supplements, from STEMCELL Technologies
StemSpan SFEM, from STEMCELL Technologies
STEMdiff™ APEL™ medium, fromSTEMCELL Technologies
X-VIVO 15, from Lonza Bioscience
XVivo 10, from Lonza Bioscience
CellGro DC, from Corning
CellGenix GMP DC, serum-free, from CellGenix
CD-U3 supplemented, from Biochrom
Small Airway Epithelial Cell Growth Medium, from PromoCell
MP-hybridoma medium, from MP Biomedicals
Human Airway Epithelial Cell (hAEC) Culture Medium, from Epithelix
SmallAir culture medium, from Epithelix
MucilAir™ Culture Medium, from Epithelix.

Besides, according to a preferred embodiment, the process of the invention is advantageously implemented without the addition of serum. Serum replacement products may be used (they are not needed when one of the media listed in the previous paragraph is used as the aqueous medium), that are not animal-sourced, including for example the followings:
KnockOut® Serum Replacement, from Gibco™
Cellastim InVitria, from Bioscience
StemSure®Serum Replacement from FUJIFILM Wako Chemical Corporation
Artificial serum Xeno-free or Animal-free from Funakoshi
Serum Replacement Solution from PeproTech
MITO+ Serum Extender from Corning

More generally, the goals of the invention may be obtained by solely making use of compounds A, B and optionally EDTA in an aqueous medium. In other words, a composition C according to the invention may advantageously not comprise other compounds.

Especially, according to preferred embodiments, the composition C does not contain all or part of the following compounds (and preferably it does not contain any of them):
gelatin for example GPS (gelatin from porcine skin), that induces more important gelling and de-gelling time;
alginates and/or pectins (that interact with multivalent ions jeopardizing the thermo-reversibility)
agarose or agar (that induces an elevation of the temperature needed for the thermo-reversibility of the hydrogel)
enzymes
serum such as FCS
antibiotics.

A composition C useful according to the instant invention has another advantage due to its composition: since compounds (A) and (B) and the optional EDTA are very easily soluble in water, the compositions C of the invention may be prepared very simply and quickly, typically by dissolving compounds (A) and (B), plus optionally EDTA, in the aqueous phase, a complete and quick dissolution being very easily obtained, for example by introducing the compounds in a powder form in the aqueous medium heated at 50-60° C. (and then cooling down the obtained composition C before contacting it with the cells).

Whatever its exact composition, a composition C according to the invention may be used, in a very simple way: especially, the obtention of a hydrogel (semi-solid composition) in STEP 2 and its liquefaction (de-gelling) in STEP 4 are only thermally induced and do not need any chemical or enzymatic reaction.

More precisely, the obtention of the hydrogel in STEP 2 is obtained by decreasing the temperature below the gelling temperature of compound C, which is typically of about 30° C.

Therefore, STEP 1 of the process wherein the composition C is used in a liquid form is typically carried out at a temperature above 30° C., for example between 3° and 60° C., and preferably between 3° and 40° C., for example between 34 and 39° C. (typically around 37° C.). And STEP 2 is typically carried out by lowering the temperature below 30° C., preferably below 20° C., for example between 1 and 15° C. (notably between 4 and 10° C.). The liquefaction (de-gelling) in STEP 4 is conversely obtained by heating the hydrogel above its liquefaction temperature which is also around 30° C. Therefore, STEP 4 of the process of the invention is typically carried out by increasing the temperature above 30° C., preferably between 3° and 40° C., for example between 34 and 39° C. (typically at 37° C.).

Typically, with the specific composition C according to the invention, the formation of the hydrogel (sol/gel transition) may be quickly obtained in STEP 2 and the liquefaction (gel/sol transition) is also relatively quick in STEP 4, with a total duration of STEP 2 plus STEP 4 that is typically below 4 hours, and in many cases below 3 hours. This constitutes a great advantage in comparison to the methods proposed in the prior art notably the method using gelatin as disclosed in U.S. Pat. No. 8,900,842 or the method using the mixture agarose/agarase of US of U.S. Pat. No. 8,709,803 since the total duration of the sol/gel plus gel/sol transitions steps is well greater with these methods (at least 5 hours).

According to interesting embodiments, STEP 2 may be carried out within less than 90 min.

The time needed in STEP 2 for forming the hydrogel generally decreases when the used temperature in STEP 2 decrease, but it also depends on the starting temperature, namely the temperature used in STEP 1. With low temperatures in STEP 2 and a temperature in STEP 1 of between 3° and 40° C., a formation of the hydrogel may typically occur within 30 to 60 min or even less, when at least around 2-3h are systematically needed with the methods of U.S. Pat. No. 8,900,842 or US of U.S. Pat. No. 8,709,803.

Likewise, according to interesting embodiments, STEP 4 of the process of the invention may be carried out within less than 150 min.

The time needed in STEP 2 for forming the hydrogel generally decreases when the used temperature in STEP 2 decrease, but it also depends on the starting temperature, namely the temperature used in STEP 1. With low temperatures in STEP 2 and a temperature in STEP 1 of between 3° and 40° C., a formation of the hydrogel may typically occur within 60 to 120 min or even less, when at least around 3-4 h are systematically needed with the methods of U.S. Pat. No. 8,900,842 or U.S. Pat. No. 8,709,803.

Given the good mechanical properties imparted by the compounds (A) and (B) a long duration may be contemplated for the transport/storage of STEP 4. Typically, STEP 4 may be carried out at least up to 36 h, which allows a long-distance shipping of the cells.

The process of the instant invention allows the transport of shipping of any kind of cells including:
isolated cells or cultured cells (including monocultures and co-cultures)
free non-adherent cells (in that first case, in contacting STEP 1, the cells are typically embedded in composition C acting as a dispersing medium for the cells) or adherent cells on a support (in that second case, in contacting STEP 1, the cells are typically coated with composition C). The support may be of any kind, including notably inserts, flasks, multiwall plates, petri dishes or other plastic ware.

According to an especially interesting embodiment, the cells transported and/or stored according to the invention are co-cultured cells adhered on an asymmetric support, typically on both faces of Transwell inserts.

The following example, corresponding to this possible embodiment, illustrates the invention.

### Example

In this example were used the following cell lines: A549, EA.hy926, THP-1 and Mo-THP-1. Their respective characteristics are as follows:
Cell line A549 corresponds to alveolar type II Human epithelial cells with ability to produce surfactant;
EA.hy926 cell line is a somatic cell hybrid with endothelial characteristics;
THP-1 is a human monocytic leukemic cell line; and
Mφ-THP-1 are macrophages derived from THP-1 cells, differentiated with PMA (Phorbol-12-myristate-13-acetate) or with 1,25-dihydroxyvitamin D3.

The cells have been used according to the following protocol:

### 1) Cell Seeding on Asymmetric Support

Cells are routinely grown in T75 flasks and trypsinized twice a week.

Medium (either in cell culturing plate, Transwell™ inserts or cell culture flasks) is changed every other day. Cells are maintained in a humidified atmosphere with 5% CO₂ at 37° C. and tested regularly for contamination by mycoplasma.

EA.hy 926 endothelial cells are seeded on inverted Transwell™ inserts (1.2×10⁵ cells/cm²; 1 μm pore size; 0.3 cm²). Upon attachment on the basolateral side of the insert, the plate is turned back to its original orientation and the epithelial cells (A549) are seeded on the top of the membrane of the Transwell™ (0.83×10⁵ cells/cm²; 0.3 cm²). Epithelial and endothelial cells are grown for three days at 37° C. with 200 μL of culture medium on the apical side and 900 μL on the basolateral one.

The used seeding medium has been prepared as followed:

Remove 62.5 mL of DMEM (Dulbecco's Modified Eagle's medium) medium from a new bottle (500 mL) and add 50 mL of FBS (10%), then 12.5 mL of HEPES stock solution (1000 mM; sterile, filtered) to get a HEPES buffered medium (25 mM)

The used Coculture medium has been prepared as followed:
Remove 12.5 mL of DMEM medium from a new bottle (500 mL) and add 12.5 mL of HEPES stock solution (1000 mM; sterile, filtered) to get a HEPES buffered medium (25 mM).
Mix and remove 125 mL
Add 50 mL of IMDM medium, then add 75 mL of RPMI+Glutamax medium

### 2) Preparation of Gelling Compositions According to the Invention

Kappa-Carrageenan from red algae (Sigma Aldrich) and carboxymethylcellulose herein-referred as “CMC” (Carboxymethylcellulose sodium salt from Sigma Aldrich-CAS Number: 9004-32-4, Product number: C5678) have been dissolved in the Co-culture medium as defined in step 1) above at different concentrations reported in the table below, leading to five compositions C1-C5 according to the invention:

| | Aqueous | kappa carrageenan | CMC |
|---|---|---|---|
| Composition | medium | content (wt %) | content (wt %) |
| C1 | Co-culture | 0.20% | 0.80% |
| C2 | medium of | 0.21% | 0.79% |
| C3 | step 1) | 0.22% | 0.78% |
| C4 | | 0.23% | 0.77% |
| C5 | | 0.24% | 0.76% |

Kappa-Carrageenan and CMC have been added in the form of powder in the medium under stirring at 60° C. When the gelling compositions obtained were still warm (>50° C.) they have been sterilized using a Sterile Vacuum Filtration System (0.22 μm pore size).

Then they have been aliquoted in appropriate volumes and supplemented with 10% of a serum replacement product. Prior to use according to the invention, the gelling composition has been brought to 37° C.

### 3) Bi-Culture Gelation (According to STEP 1 and STEP 2 of the Invention)

The cell culture medium was removed from both the sides of the Transwell™ inserts and was replaced with the composition C1 as described in paragraph 2) and kept at 37° C. in a water-bath until use (300 μL on the apical side and 900 μL on the basolateral side). Compositions C2 to C5 may be used in the same way.

The temperature was let cool down for 60 min at 17-21° C. keeping the multiwell plate open under the laminar flow hood, then the plate was closed and sealed.

### 4) Transport/Storage of the Gelled Bi-Culture (According to STEP 3 of the Invention)

The obtained gelled plates were transported in a refrigerated box maintained at a temperature between 4° C.-10° C. for 24 hours.

### 5) Bi-Culture De-Gelling (According to STEP 4 of the Invention)

The sealing was removed and the gelled bi-culture was put in an incubator (37° C., 5% CO₂, 95% humidity) for 2 h. The liquefied composition was removed by pipetting and both the sides of the inserts were washed three times with cell culture medium to remove potential residues of gel.

### 6) Tri-Culture Assembling

A triculture was prepared from the de-gelled bi-culture according to the following protocol:

Detach the macrophage-like cells previously differentiated from THP-1 with PMA.

Prepare a cell suspension at a density of 1.8×10⁵ cells/mL in cell culture medium. Put 200 μL of this suspension on the apical side of each inserts after removal of the gel and 900 L of medium on the basolateral side. After 4 h the macrophages should be attached; remove completely the medium from the apical side and reduce the medium on the basolateral side to 200 μL (producing ALI conditions).

1. A process for the in vitro storing and/or transporting of cells, comprising the following steps:
STEP 1: contacting the cells with an aqueous thermo-reversible gelling composition C, said composition C being in a liquid form and comprising, in an aqueous medium:
(B) a thermo-reversible gelling polysaccharide selected from carrageenan, gellan gum and konjac gum; and (B) carboxymethyl cellulose as a thickening agent;
STEP 2: thermo-gelling the composition C in contact with the cells as obtained in step 1, by decreasing the temperature, whereby a hydrogel is obtained, embedding the cells; and
STEP 3: storing and/or transporting the cells embedded in the hydrogel as obtained in step 2.

2. The process of claim 1, further comprising the following additional step 4, after step 3:
STEP 4: liquefying the hydrogel by increasing the temperature, whereby the cells are released from the hydrogel.

3. The process of claim 1, wherein the aqueous medium of composition C is a culture medium suitable for the stored and/or transported cells.

4. The process of claim 1, wherein STEP 1 is carried out at a temperature between 3° and 60° C.

5. The process of claim 1, wherein STEP 2 is carried out by lowering the temperature below 30° C.

6. The process of claim 2, wherein STEP 4 is carried out at a temperature between 3° and 40° C.

7. The process of claim 2, wherein the total duration of STEP 2 plus STEP 4 is below 4 hours.

8. A composition C suitable for the process of claim 1, comprising, in an aqueous medium:
(A) a thermo-reversible gelling polysaccharide selected from carrageenan, gellan gum and konjac gum; and
(B) carboxymethyl cellulose as a thickening agent

9. The composition of claim 8, wherein the compound (A) includes a carrageenan.

10. The composition of claim 8, wherein the compound (B) is a carboxymethyl cellulose having a viscosity lower than 40 mPa·s at 25° C. at 2% by weight in water.

11. The composition of claim 8, wherein the ratio A/B of the total mass of the compound (A) to the total mass of the compound (B) is between 15:85 and 40:60.

12. The composition of claim 8, wherein the total concentration of the compounds (A) and (B), namely the ratio of the sum of the mass of compound (A) plus the mass of compound (B) to the total volume of composition (C) is between 6 to 10 g/L.

13. The composition of claim 8, which further comprises ethylenediaminetetraacetic acid (EDTA) as a chelating agent.

14. The composition of claim 8, which do not contain any substances from animal origin.

15. The process of claim 4, wherein STEP 1 is carried out at a temperature between 3° and 40° C.

16. The process of claim 5, wherein STEP 2 is carried out by lowering the temperature between 1 and 15° C.

17. The process of claim 6, wherein STEP 4 is carried out at a temperature between 34 and 39° C.

18. The process of claim 7, wherein the total duration of STEP 2 plus STEP 4 is below 3 hours.

19. The composition of claim 8, wherein the compound (A) includes a kappa-carrageenan.

20. The composition of claim 12, wherein the total concentration of the compounds (A) and (B), namely the ratio of the sum of the mass of compound (A) plus the mass of compound (B) to the total volume of composition (C) is between 8 to 10 g/L.

21. The composition of claim 13, which further comprises ethylenediaminetetraacetic acid (EDTA) as a chelating agent at a content of less than 1 mM.
